(19) United States
(12) Patent Application Publication
(10) Pub. No.: US 2011/0144460 A1
(43) Pub. Date: Jun. 16, 2011

(54) PULSE FREQUENCY MEASURING METHOD AND APPARATUS

(75) Inventors: Jung-Taek OH, Seoul (KR); Young-Chul Park, Wonju-si (KR); Se-Dong Min, Seoul (KR); Min-Hyoung Lee, Yongin-si (KR)

(73) Assignees: Samsung Electronics Co., Ltd.; Industry-Academic Cooperation Foundation Yonsei University

(21) Appl. No.: 12/967,661

(22) Filed: Dec. 14, 2010

(30) Foreign Application Priority Data
Dec. 14, 2009 (KR) 10-2009-0124072

(51) Int. Cl. A61B 5/1455; A61B 5/0452; G06F 19/00

(52) U.S. Cl. 600/324; 600/509; 702/19

(57) ABSTRACT

A method and an apparatus are provided for measuring a pulse frequency in a bio-signal measurement device. A bio-signal collected by a sensor is applied as an input signal of a notch filter. A filter coefficient of the notch filter is adaptively changed according to a result of tracking the bio-signal in the notch filter and calculating a pulse frequency corresponding to the filter coefficient of the notch filter.

### PRIORITY

This application claims priority under 35 U.S.C. §119(a) to an application entitled “Pulse Frequency Measuring Method and Apparatus” filed in the Korean Intellectual Property Office on Dec. 14, 2009 and assigned Serial No. 10-2009-0124072, the contents of which are incorporated herein by reference.

### BACKGROUND OF THE INVENTION

1. Field of the Invention

The present invention relates generally to a pulse frequency measuring method and apparatus, and more particularly, to a method for measuring a correct pulse frequency when noise is included in a bio-signal and an apparatus therefor.

2. Description of the Related Art

A living-body examination device provides (and/or displays) various pieces of living-body information regarding a subject of the living-body (e.g., the heart of a human body) in a form recognizable to a predetermined examiner by collecting and analyzing a minute action current generated in the subject, e.g., an electrical change of the action current, etc. For example, a living-body examination device connects a measuring electrode to a subject that is to be examined, collects a bio-signal, such as an ElectroCardioGram (ECG) or a PhotoPlethysmoGraphy (PPG), by analyzing a change of a voltage induced to the measuring electrode, and estimates and provides a pulse frequency using the collected bio-signal.

In order to measure a bio-signal, a living-body examination device must physically connect a measuring electrode to a surface of a subject. However, because the subject may continuously move or because the measuring electrode may be disconnected from a set measuring point, an impedance change between the subject and the measuring electrode is inevitable.

The impedance change may act as noise, e.g., user motion artifacts or sensor contact noise, against the bio-signal collected by the living-body examination device, thereby distorting a waveform of the measured bio-signal, and thus, deriving a wrong result. For example, a pulse frequency is calculated using a peak interval of a bio-signal associated with a pulse, and if noise is repeated on the bio-signal, correct peak values of the bio-signal cannot be calculated. Accordingly, an incorrect pulse frequency may be calculated, thereby causing an error.

### SUMMARY OF THE INVENTION

The present invention has been made to address at least the above problems and/or disadvantages and to provide at least the advantages described below. Accordingly, an aspect of the present invention provides a method and an apparatus for detecting a correct bio-signal in any surrounding environment.

Another aspect of the present invention provides a method and an apparatus for measuring a correct pulse frequency when noise is included in a bio-signal.

A further aspect of the present invention provides a method and an apparatus for compensating for a degeneration period, which may occur in a bio-signal processing process.

According to one aspect of the present invention, a method is provided for measuring a pulse frequency in a bio-signal measurement device. A bio-signal collected by a sensor is applied as an input signal of a notch filter. A filter coefficient of the notch filter is adaptively changed according to a result of tracking the bio-signal in the notch filter and a pulse frequency is calculated corresponding to the filter coefficient of the notch filter.

According to another aspect of the present invention, an apparatus is provided for measuring a pulse frequency in a bio-signal measurement system. The apparatus comprises a bio-signal processor for adaptively changing a filter coefficient of a notch filter according to a result of tracking a bio-signal in the notch filter when the bio-signal collected by a sensor is applied as an input signal of the notch filter, and for calculating a pulse frequency corresponding to the filter coefficient of the notch filter. The apparatus also comprises a display unit for displaying the pulse frequency output from the bio-signal processor.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The above and other aspects, features and advantages of the present invention will become more apparent from the following detailed description when taken in conjunction with the accompanying drawing in which:

FIG. 1 is a block diagram illustrating a bio-signal measurement device, according to an embodiment of the present invention;

FIG. 2 is a block diagram illustrating a degeneration period detector, according to an embodiment of the present invention;

FIG. 3 is a block diagram illustrating an impulse noise detector, according to an embodiment of the present invention;

FIG. 4 is a flowchart illustrating an operation of the bio-signal measurement device, according to an embodiment of the present invention;

FIG. 5 is a flowchart illustrating an operation of the degeneration period detector, according to an embodiment of the present invention;

FIG. 6 is a flowchart illustrating an operation of the impulse noise detector, according to an embodiment of the present invention; and

FIGS. 7 and 8 illustrate bio-signals tracked by a notch filter, according to an embodiment of the present invention.

### DETAILED DESCRIPTION OF EMBODIMENTS OF THE PRESENT INVENTION

Embodiments of the present invention are described in detail with reference to the accompanying drawings. The same or similar elements may be denoted by the same or similar reference numerals even though they are depicted in different drawings. Detailed descriptions of constructions or processes known in the art may be omitted to avoid obscuring the subject matter of the present invention.

Conventional methods of measuring a pulse frequency through continuous monitoring of a bio-signal, and measuring a pulse frequency from a time of a peak interval of a bio-signal may provide an incorrect result due to an influence of a noise environment, such as user motion artifacts, sensor contact noise, etc., which occur during an exercise.

Embodiments of the present invention detect a more correct pulse frequency by using a notch filter to track a bio-signal, perceive a signal period in which noise exists or an abnormal signal period, and properly estimate a bio-signal in the corresponding period.

According to an embodiment of the present invention, a bio-signal measurement device converts a notch frequency into a pulse frequency. The notch frequency is obtained from a filter coefficient estimated by performing a pre-processing process in which adaptive filtering for removing a motion artifact signal included in a detected bio-signal is performed. The pre-processed bio-signal is input to a notch filter, e.g., second-order Infinite Impulse Response (IIR) adaptive notch filter, and a filter coefficient of the notch filter is adaptively updated.

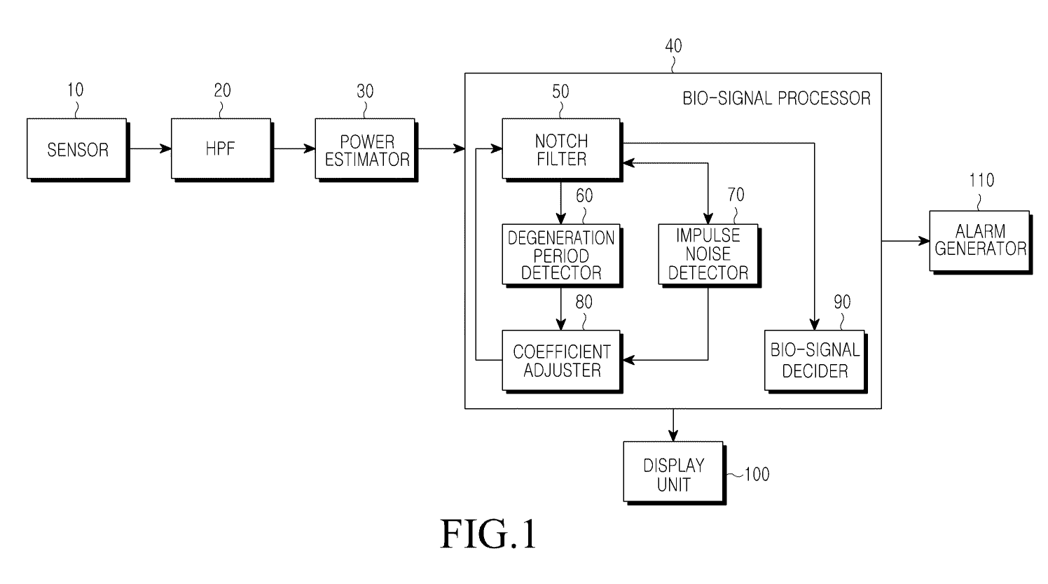

FIG. 1 is a block diagram illustrating the bio-signal measurement device, according to an embodiment of the present invention. The bio-signal measurement device includes a sensor 10, a High Pass Filter (HPF) 20, and a power estimator 30 for performing a pre-processing process. The bio-signal measurement device also includes a bio-signal processor 40, a display unit 100, and an alarm generator 110.

The sensor 10 generates a bio-signal by collecting a minute action current generated by a subject (e.g., the heart of a human body), an electrical change of the action current, etc. The action current or electrical change is converted to an electrical signal. The bio-signal is output to the HPF 20.

The HPF 20 is an adaptive filter for removing a motion artifact signal included in the input bio-signal. The motion artifacts may be generated due to a motion of a user, such as breathing. Since the motion artifact signal has a low frequency, the motion artifact signal can be removed by the HPF 20. The bio-signal filtered by the HPF 20 is input to the power estimator 30.

The power estimator 30 estimates power of the input bio-signal. If the estimated power is equal to or greater than a minimum value, the power estimator 30 outputs the input bio-signal to the bio-signal processor 40. Otherwise, the power estimator 30 ignores the input bio-signal. A signal input to the power estimator 30 is an invalid signal unless the input signal has power equal to or greater than the minimum value.

The display unit 100 displays a bio-signal or bio-information input from the bio-signal processor 40.

The bio-signal processor 40 tracks a bio-signal by using a notch filter. A signal of a period degenerated in a pre-processing process through the HPF 20 or degenerated due to physical reasons in a process of collecting the bio-signal is estimated and restored. A final bio-signal is determined by tracking only the bio-signal in a noise period remaining in the bio-signal. Bio-information, such as a pulse frequency, is calculated by using the final bio-signal, and the bio-information is output to the display unit 100.

The bio-signal processor 40 includes a notch filter 50, a degeneration period detector 60, an impulse noise detector 70, a coefficient adjuster 80, and a bio-signal decider 90.

Since the notch filter 50 is able to track a mono-frequency corresponding to an input signal and notch the tracked frequency, the notch filter 50 is suitable for tracking a frequency of a bio-signal, e.g., an ECG signal or a PPG signal, having mono-frequency characteristics. In addition, since a filter coefficient of the notch filter 50 determines a notch frequency and is proportional to a frequency of the input signal, a frequency of a bio-signal can be perceived by using the filter coefficient. Accordingly, a pulse frequency can be easily calculated. Thus, the bio-signal decider 90 preferably includes an adaptive notch filter, e.g., a second-order IIR adaptive notch filter.

According to an embodiment of the present invention, the bio-signal output from the power estimator 30 becomes an input signal of the notch filter 50. If the bio-signal is input, the notch filter 50 estimates a frequency of the bio-signal by tracking the input bio-signal and filters the input bio-signal by adaptively setting a filter coefficient thereof according to the estimated frequency. A tracking speed of the notch filter 50 on the input signal is determined by the coefficient adjuster 80. The notch filter 50 outputs the tracked bio-signal to the impulse noise detector 70 and outputs the filtered signal to the degeneration period detector 60.

In the pre-processing process in which a motion artifact signal included in a bio-signal is removed, partial periods of the bio-signal may be lost due to motion artifacts, or the bio-signal may be discontinuously collected by the sensor 10. The notch filter 50 loses an input signal to be tracked, thereby diverging or significantly fluctuating. Meanwhile, impulse noise instantaneously having great energy may be introduced into the bio-signal. A frequency band of the impulse noise overlaps a frequency band of the bio-signal. Since energy of the impulse noise is much greater than that of the bio-signal, the notch filter 50 may track a frequency of the impulse noise instead of the bio-signal.

Thus, in an embodiment of the present invention, the degeneration period detector 60 determines a degeneration period in which the bio-signal is lost, by comparing power of an input signal of the notch filter 50 with power of an output signal of the notch filter 50. The impulse noise detector 70 determines an impulse noise period in which impulse noise having great energy is introduced, by comparing signals envelope-estimated by applying different attack times to power of an input signal tracked by the notch filter 50, i.e., a tracked bio-signal. The degeneration period detector 60 or the impulse noise detector 70 informs the coefficient adjuster 80 of whether a corresponding signal period is a degeneration period or an impulse noise period.

If the degeneration period or the impulse noise period is informed, the coefficient adjuster 80 determines a tracking coefficient of the notch filter 50 so that an input signal tracking speed of the notch filter 50 decreases, and sets the determined tracking coefficient to the notch filter 50. The coefficient adjuster 80 outputs information regarding the degeneration period and the impulse noise period to the bio-signal decider 90 to decide a final bio-signal and bio-information.

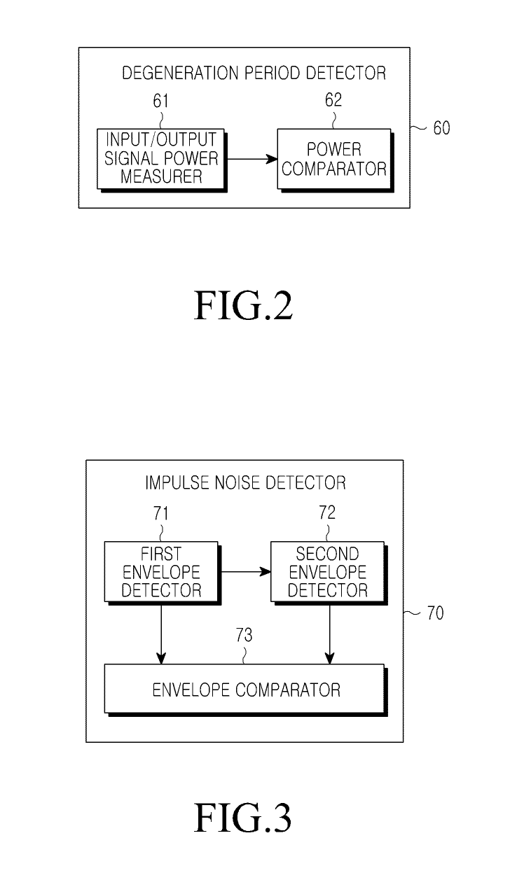

Embodiments of the degeneration period detector 60 and the impulse noise detector 70 are shown in FIGS. 2 and 3, respectively. FIG. 2 is a block diagram illustrating the degeneration period detector 60, according to an embodiment of the present invention. The degeneration period detector 60 includes an input/output signal power measurer 61 and a power comparator 62.

The input/output signal power measurer 61 measures power of an input signal input to the notch filter 50 and power of an output signal output from the notch filter 50, and delivers the power of the input signal and the power of the output signal to the power comparator 62.

If it is assumed that background noise exists in the form of white noise without any signal, since a mono frequency that the notch filter 50 must track does not exist, the notch filter 50 diverges or tracks a frequency in a wrong direction. On the contrary, if it is assumed that background noise exists in the form of colored noise, the notch filter 50 tracks a frequency of the colored noise, wherein energy of the colored noise is not generally great. Thus, if it is assumed that the notch filter 50 ideally notches only a bio-signal, a ratio of the power of an input signal input to the notch filter 50 to the power of an output signal output from the notch filter 50 in a degeneration period in which no bio-signal exists will typically be approximately 1.

Accordingly, the power comparator 62 compares the power of the input signal with the power of the output signal. A corresponding period is determined as a degeneration period if a difference between the power of the input signal and the power of the output signal is less than a predetermined power reference value. Specifically, when a ratio of the power of the output signal to the power of the input signal is less than the predetermined power reference value, the corresponding period is determined to be a degeneration period. Equation (1) is used to calculate the ratio of the power of the output signal to the power of the input signal according to an embodiment of the present invention.

$$ratio = \frac{{P}_{y}}{{P}_{x}} , \\ {P}_{x} = E [ {x}^{2} ] , {P}_{y} = E [ {y}^{2} ] \qquad ( 1 )$$

P_{y} denotes power of an output signal, Pₓ denotes power of an input signal, x denotes an input signal of the notch filter 50, and y denotes an output signal of the notch filter 50.

However, since an ensemble average cannot be obtained in actual implementation, a ratio of the power of the output signal to the power of the input signal, which is calculated by estimation with an IIR average can be represented by Equations (2) to (4).

$$\begin{matrix}{\ddot{P}}_{x} = λ {\ddot{P}}_{x} ( n - 1 ) + ( 1 - λ ) {x}^{2} ( n ) & ( 2 ) \\ {\ddot{P}}_{y} = λ {\ddot{P}}_{y} ( n - 1 ) + ( 1 - λ ) {y}^{2} ( n ) & ( 3 ) \\ ratio = \frac{{\ddot{P}}_{y}}{{\ddot{P}}_{x}} & ( 4 )\end{matrix}$$

\ddot{P}^{y}ₓ denotes power of an estimated input signal, I^{y}_{y} denotes power of an estimated output signal, λ denotes a smoothing factor for power estimation, x(n) denotes an input signal of the notch filter 50, y(n) denotes an output signal of the notch filter 50, ratio denotes an estimated power ratio of an output signal to an input signal, which has a value between 0 and 1.

Accordingly, the power reference value can be defined as a minimum value of a ratio of power of an input signal to power of an output signal, which can be calculated when a bio-signal exists.

Since the adaptive dual notch filter 50 tracks a frequency band having great energy, if impulse noise having greater energy than a bio-signal is introduced, it is difficult for the notch filter 50 to track the bio-signal, and the notch filter 50 tracks the impulse noise. Accordingly, a pulse frequency greater or less than a pulse frequency of an actual user may be instantaneously calculated in an impulse noise period. However, since the notch filter 50 must consistently provide a pulse frequency, the impulse noise period must be determined.

Since impulse noise is generated by instantaneously introducing an unexpected signal having great energy, the impulse noise is represented as a signal having a relatively greater value than a main signal during a short time in a time domain and is represented as a signal spread in a wide frequency band in a frequency domain. If impulse noise is introduced, a bio-signal, i.e., the input signal of the notch filter 50, instantaneously has great energy, and power of the input signal is rapidly changed. Embodiments of the present invention determine an impulse noise period using these characteristics.

FIG. 3 is a block diagram illustrating the impulse noise detector 70, according to an embodiment of the present invention. The impulse noise detector 70 determines an impulse noise period of a bio-signal by comparing signals envelope-estimated by applying different attack times to power of an input signal tracked by the notch filter 50.

Specifically, the impulse noise detector 70 estimates two envelopes for power of an input signal by envelope-estimating power of a tracked input signal input from the notch filter 50 using two different attack time constants. The impulse noise detector 70 also determines an impulse noise period by using a ratio of two estimated envelopes. An envelope to which a fast attack time constant of the two different attack time constants is applied has a rapid change width at the beginning of the impulse noise period, while an envelope to which a slow attack time constant is applied has a relatively narrow change width in the impulse noise period. Thus, a difference between the two envelopes is greater than a predetermined reference value in a period in which impulse noise exists. Embodiments of the present invention determine an impulse noise period by using this characteristic. In order to make a duration of the impulse noise period the same, the same release time is applied to estimate each envelope.

The impulse noise detector 70 includes a first envelope detector 71, a second envelope detector 72, and an envelope comparator 73, as shown in FIG. 3. The first envelope detector 71 estimates an envelope to which a first attack time constant is applied for the power of the input signal tracked by the notch filter 50. The second envelope detector 72 estimates an envelope to which a second attack time constant is applied for the power of the input signal tracked by the notch filter 50. It is assumed that the first attack time constant is a time constant having a faster attack time than the second attack time constant. The first envelope detector 71 and the second envelope detector 72 output the estimated envelopes to the envelope comparator 73. The envelope comparator 73 compares a ratio of the second envelope to the first envelope with a predetermined envelope reference value. If the ratio of the second envelope to the first envelope is less than the predetermined envelope reference value, the envelope comparator 73 determines a corresponding period as an impulse noise period and informs the coefficient adjuster 80 of this determination.

With the above-described process, the coefficient adjuster 80 can receive whether the input signal of the notch filter 50, i.e., a bio-signal, corresponds to a degeneration period or impulse noise period. Accordingly, the coefficient adjuster 80 decides a tracking coefficient for defining an estimated speed of the input signal of the notch filter 50 and sets the decided tracking coefficient to the notch filter 50.

More specifically, if the coefficient adjuster 80 receives from the degeneration period detector 60 and the impulse noise detector 70 that a current input signal of the notch filter 50 is in a normal state, the coefficient adjuster 80 maintains a tracking coefficient of the notch filter 50 as a standard value. However, if the coefficient adjuster 80 receives from the degeneration period detector 60 and the impulse noise detector 70 that a current input signal of the notch filter 50 corresponds to a degeneration period or an impulse noise period, the coefficient adjuster 80 decides a tracking coefficient so that a tracking speed of the input signal decreases, and sets the decided tracking coefficient to the notch filter 50. Thereafter, the coefficient adjuster 80 outputs information regarding the degeneration period and the impulse noise period to the bio-signal decider 90.

A decrease of a tracking speed of the notch filter 50 in an abnormal signal period can prevent the notch filter 50 from diverging and prevent unconditional tracking for impulse noise, thereby making tracking approximate to a bio-signal possible.

The bio-signal decider 90 perceives the filter coefficient of the notch filter 50 in real-time, acquires a notch frequency from the filter coefficient, calculates a pulse frequency by using the acquired notch frequency, and outputs the calculated pulse frequency to the display unit 100. However, if a bio-signal corresponds to a degeneration period, the bio-signal decider 90 calculates a pulse frequency by using filter coefficients detected during a predetermined period before the degeneration period until the bio-signal is in the normal state again. Accordingly, the bio-signal decider 90 stores and updates filter coefficients detected during a recent normal state for a predetermined period. During the degeneration period, the bio-signal decider 90 controls the notch filter 50 to maintain the filter coefficient of the notch filter 50 as a filter coefficient used to calculate a pulse frequency for a non-degeneration period. In addition, the bio-signal decider 90 controls the alarm generator 110 to generate an alarm sound for alarming that a currently calculated pulse frequency may be incorrect, for the degeneration period and the impulse noise period. According to another embodiment of the present invention, the notch filter 50 may be controlled to simply delay a tracking speed for an input signal without fixing the filter coefficient of the notch filter 50 in a degeneration period. Only if the degeneration period is not continuous over a predetermined period of time, a schematic degeneration period can be estimated and the notch filter 50 can quickly track a bio-signal when the bio-signal exists again.

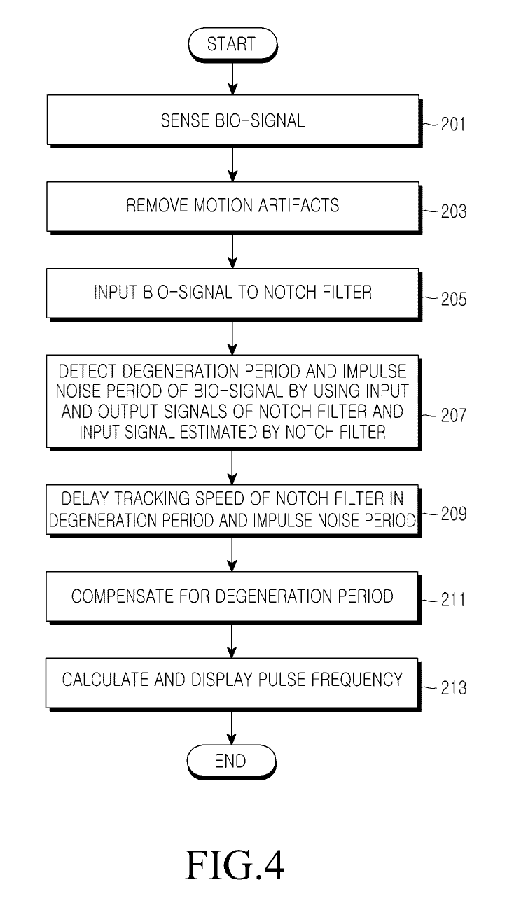

An operation of the bio-signal measurement device as described above is illustrated in FIG. 4, according to an embodiment of the present invention. The bio-signal measurement device generates a bio-signal through the sensor 10 in step 201 and removes motion artifacts by delivering the bio-signal to the HPF 20 in step 203. In step 205, the bio-signal measurement device inputs the bio-signal from which the motion artifacts are removed to the bio-signal processor 40 through the power estimator 30 so that the bio-signal is an input signal of the notch filter 50.

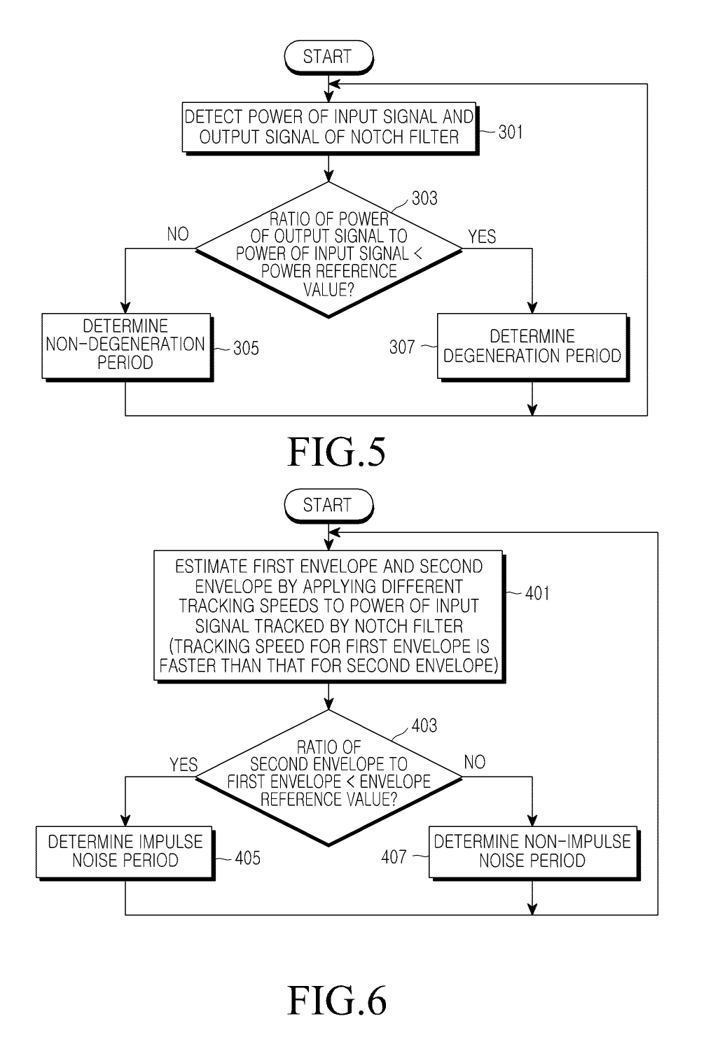

In step 207, the bio-signal processor 40 of the bio-signal measurement device detects a degeneration period and an impulse noise period by using input and output signals of the notch filter 50 and an input signal estimated by the notch filter 50. Processes of detecting the degeneration period and the impulse noise period are illustrated in FIGS. 5 and 6, respectively. FIG. 5 is a flowchart illustrating a degeneration period detection operation of the degeneration period detector 60, according to an embodiment of the present invention. FIG. 6 is a flowchart illustrating an impulse noise period detection operation of the impulse noise detector 70, according to an embodiment of the present invention.

Referring to FIG. 5, the degeneration period detector 60 detects power of the input signal of the notch filter 50 and power of the output signal of the notch filter 50 in step 301. The degeneration period detector 60 compares a ratio of the power of the output signal to the power of the input signal with a power reference value in step 303. If the power ratio is less than the power reference value, the degeneration period detector 60 determines in step 307 that a current input signal corresponds to a degeneration period. If the power ratio is greater than or equal to the power reference value, the degeneration period detector 60 determines in step 305 that the current input signal corresponds to a non-degeneration period.

Referring to FIG. 6, the impulse noise detector 70 estimates a first envelope and a second envelope by changing a tracking speed for power of the input signal estimated by the notch filter 50 in step 401. It is assumed that a tracking speed for the first envelope is faster than that for the second envelope. The impulse noise detector 70 compares a ratio of the second envelope to the first envelope with an envelope reference value in step 403. If the envelope ratio is less than the envelope reference value, the impulse noise detector 70 determines in step 405 that a current input signal corresponds to an impulse noise period. If the envelope ratio is greater than or equal to the envelope reference value, the impulse noise detector 70 determines in step 407 that the current input signal corresponds to a non-impulse noise period.

Referring back to FIG. 4, if the degeneration period or the impulse noise period are detected by the processes as shown in FIGS. 5 and 6, the bio-signal processor 40 delays a tracking speed for the input signal of the notch filter 50 by a predetermined unit in step 209. In step 211, the bio-signal processor 40 decides a final bio-signal by estimating a degeneration period using a filter coefficient of the notch filter 50 detected in a normal period immediately before the degeneration period.

In step 213, the bio-signal processor 40 calculates a pulse frequency by using the filter coefficient of the notch filter 50 and displays the pulse frequency on the display unit 100 as bio-information.

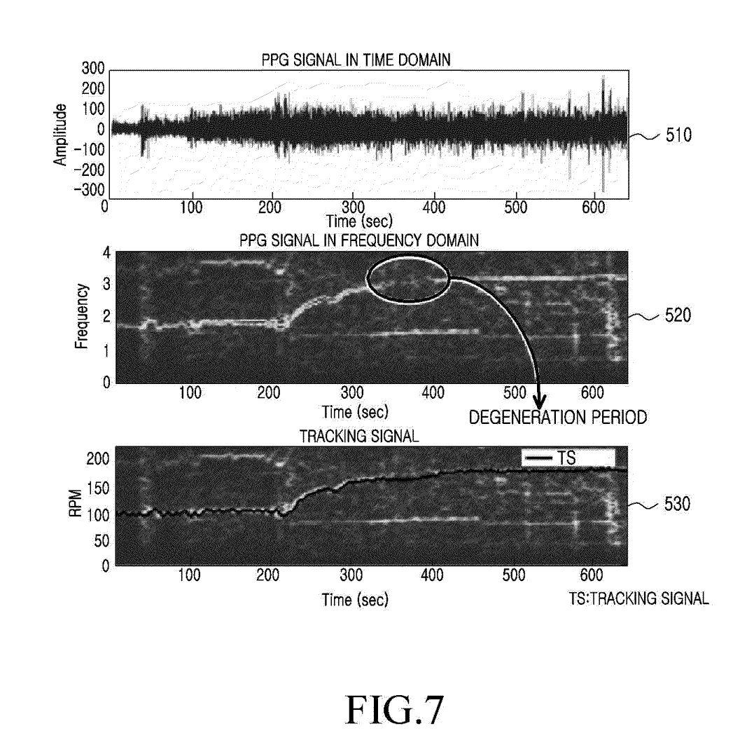

FIG. 7 illustrates a bio-signal that is determined by applying an embodiment the present invention to a degeneration period in which a PPG signal is removed together when motion artifacts are removed from the PPG signal. In FIG. 7, a first waveform diagram 510 indicates a time domain of a PPG signal in which motion artifacts overlap. A second waveform diagram 520 indicates a frequency domain of a PPG signal removed when the motion artifacts are removed. A third waveform diagram 530 indicates a frequency domain of a PPG signal finally tracked by estimating a degeneration period, according to an embodiment of the present invention. Referring to the second waveform diagram 520, it can be observed in the frequency domain that a PPG frequency is lost from around 330 seconds to 400 seconds. However, the PPG signal is tracked and the degeneration period is estimated according to the present invention, as shown in the third waveform diagram 530, by tracking the PPG signal in a normal period and delimiting the PPG signal in a degeneration period so that the PPG signal is not largely out of a previously tracked PPG frequency. Accordingly, the degeneration period can be estimated, and the notch filter 50 can quickly track the PPG signal when the PPG signal exists again.

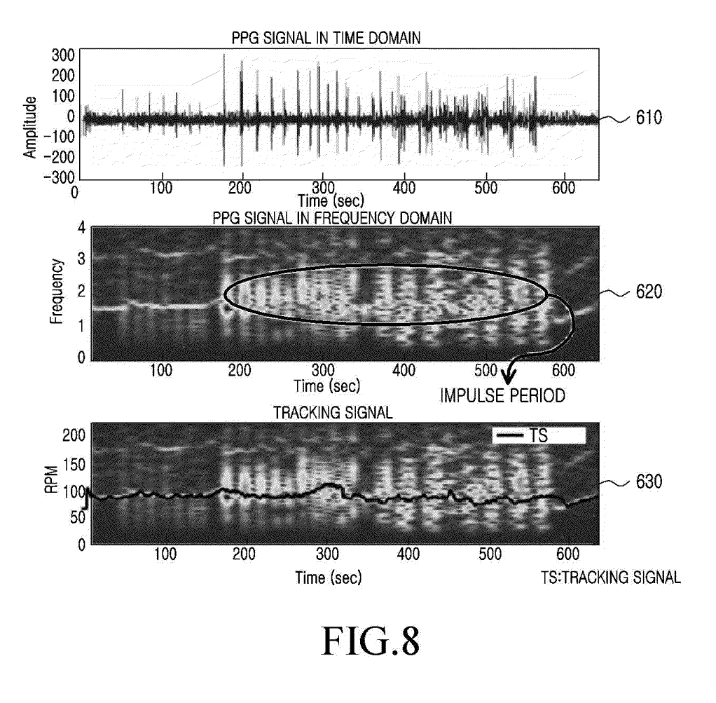

FIG. 8 illustrates a bio-signal that is determined by applying an embodiment of the present invention to a state in which a PPG signal is introduced with impulse noise. In FIG. 8, a fourth waveform diagram 610 indicates a time domain of a PPG signal in which impulse noise overlaps. A fifth waveform diagram 620 indicates a frequency domain of the PPG signal in which the impulse noise overlaps. A sixth waveform diagram 630 indicates a frequency domain of a PPG signal finally tracked by estimating an impulse noise period, according to an embodiment of the present invention. Referring to the sixth waveform diagram 630, it can be confirmed that a bio-signal is stably tracked even in an impulse noise period in which an energy change is rapid due to impulse noise.

While the invention has been shown and described with reference to a certain embodiments thereof, it will be understood by those skilled in the art that various changes in form and detail may be made therein without departing from the spirit and scope of the invention as defined by the appended claims.

What is claimed is:

1. A method for measuring a pulse frequency in a bio-signal measurement device, the method comprising the steps of:
applying a bio-signal collected by a sensor as an input signal of a notch filter; and
adaptively changing a filter coefficient of the notch filter according to a result of tracking the bio-signal in the notch filter and calculating a pulse frequency corresponding to the filter coefficient of the notch filter.

2. The method of claim 1, wherein calculating a pulse frequency comprises:
determining a degeneration period of the bio-signal by using the input signal and an output signal of the notch filter;
determining an impulse noise period in which an impulse noise signal is introduced, by measuring a power of the bio-signal tracked by the notch filter;
delaying a tracking speed of the notch filter in the degeneration period and the impulse noise period; and
calculating the pulse frequency corresponding to the filter coefficient of the notch filter.

3. The method of claim 2, wherein determining a degeneration period of the bio-signal comprises determining a corresponding period as a degeneration period when a difference between a power of the input signal and a power of the output signal is less than a predetermined power reference value and continuously maintaining a filter coefficient from before the degeneration period during the degeneration period.

4. The method of claim 3, wherein the filter coefficient of the notch filter detected in a normal period of the bio-signal tracked by the notch filter is stored and updated during a predetermined period.

5. The method of claim 2, wherein the impulse noise period is determined by comparing two signals that are envelope-estimated by applying different attack time constants to the power of the bio-signal tracked by the notch filter.

6. The method of claim 5, wherein determining an impulse noise period comprises:
detecting a first envelope envelope-estimated by applying a first attack time constant to the power of the bio-signal tracked by the notch filter;
detecting a second envelope envelope-estimated by applying a second attack time constant, which is slower than the first attack time constant, to the power of the bio-signal tracked by the notch filter; and
determining the impulse noise period, when a ratio of the second envelope to the first envelope is less than an envelope reference value.

7. The method of claim 1, wherein, before the bio-signal collected by the sensor is applied to the notch filter, motion artifacts are removed from the bio-signal by a High Pass Filter (HPF).

8. The method of claims 1, wherein the bio-signal is one of an ElectroCardioGram (ECG) and a PhotoPlethysmoGraphy (PPG).

9. An apparatus for measuring a pulse frequency in a bio-signal measurement system, the apparatus comprising:
a bio-signal processor for adaptively changing a filter coefficient of a notch filter according to a result of tracking a bio-signal in the notch filter when the bio-signal collected by a sensor is applied as an input signal of the notch filter, and for calculating a pulse frequency corresponding to the filter coefficient of the notch filter; and
a display unit for displaying the pulse frequency output from the bio-signal processor.

10. The apparatus of claim 9, wherein the bio-signal processor comprises:
a degeneration period detector for determining a degeneration period of the bio-signal by using the input signal and an output signal of the notch filter;
an impulse noise detector for determining an impulse noise period in which an impulse noise signal is introduced, by measuring a power of the bio-signal tracked by the notch filter;
a coefficient adjuster for delaying a tracking speed of the notch filter in the degeneration period and the impulse noise period; and
a bio-signal decider for calculating the pulse frequency corresponding to the filter coefficient of the notch filter.

11. The apparatus of claim 10, wherein the degeneration period detector determines a corresponding period as a degeneration period when a difference between a power of the input signal and a power of the output signal is less than a predetermined power reference value, and continuously maintains a filter coefficient from before the degeneration period during the degeneration period.

12. The apparatus of claim 11, wherein the bio-signal decider controls the notch filter to continuously maintain the filter coefficient from before the degeneration period during the degeneration period

13. The apparatus of claim 12, wherein the filter coefficient of the notch filter detected in a normal period of the bio-signal tracked by the notch filter is stored and updated during a predetermined period.

14. The apparatus of claim 10, wherein the impulse noise detector determines the impulse noise period by comparing two signals that are envelope-estimated by applying different attack time constants to the power of the bio-signal tracked by the notch filter.

15. The apparatus of claim 14, wherein the impulse noise detector detects a first envelope envelope-estimated by applying a first attack time constant to the power of the bio-signal tracked by the notch filter, detects a second envelope envelope-estimated by applying a second attack time constant, which is slower than the first attack time constant, to the power of the bio-signal tracked by the notch filter, and determines the impulse noise period when a ratio of the second envelope to the first envelope is less than an envelope reference value.

16. The apparatus of claim 9, further comprising a High Pass Filter (HPF) for removing motion artifacts from the bio-signal collected by the sensor and applying the bio-signal to the notch filter.

17. The apparatus of claim 9, wherein the bio-signal is one of an ElectroCardioGram (ECG) and a PhotoPlethysmoGraphy (PPG).
